(19) United States
(12) Patent Application Publication
(10) Pub. No.: US 2017/0135736 A1
(43) Pub. Date: May 18, 2017

(54) SURGICAL WRENCH

(71) Applicant: Spinal Simplicity, LLC, Overland Park, KS (US)

(72) Inventors: Adam Frock, Larwill, IN (US); Jeff Slover, Lee's Summit, MO (US); Douglas Snell, Overland Park, KS (US); Luke Luallin, Leawood, KS (US); Todd Moseley, Olathe, KS (US)

(73) Assignee: Spinal Simplicity, LLC, Overland Park, KS (US)

(21) Appl. No.: 15/356,095

(22) Filed: Nov. 18, 2016

Related U.S. Application Data
(60) Provisional application No. 62/257,065, filed on Nov. 18, 2015.

(51) Int. Cl. A61B 17/70

(52) U.S. Cl. CPC A61B 17/7082; A61B 17/8875

(57) ABSTRACT

A surgical wrench includes a longitudinal body having a central axis, a proximal portion and an opposing distal portion. The longitudinal body includes an inner shaft and an outer shaft surrounding at least a portion of the inner shaft. A removable adaptor is operatively connected to the distal portion including a ratchet mechanism configured to rotate a surgical implant. A plunger is operatively associated with a proximal end of the inner shaft configured to translate the inner shaft along the central axis to engage or disengage the surgical implant.

### CROSS-REFERENCE TO RELATED APPLICATION

The subject invention claims the benefit of and priority to U.S. Provisional Patent Application Ser. No. 62/257065 filed Nov. 18, 2015, the disclosure of which is herein incorporated by reference in its entirety.

### BACKGROUND OF THE INVENTION

1. Field of the Invention

This invention relates generally to medical instruments and more particularly to a wrench used for surgical implants deployed in a patient's body.

2. Description of Related Art

When performing surgical operations and procedures, including orthopedic and trauma related procedures, time and accuracy is of the essence. Orthopedic, trauma and other surgical tools, instruments and implants may require tightening, loosening or other types of adjustments during the surgical procedure.

For example, interspinous process decompression (IPD) is a less invasive surgical procedure for treating spinal stenosis. With IPD surgery, there is no removal of bone or soft tissue. Instead, an implant or spacer device is positioned behind the spinal cord or nerves between the interspinous processes that protrude from the vertebrae in the lower back. Typically in these types of surgical procedures, it is necessary to tighten a bolt or other type of fastening, retaining or other type of adjustment mechanism to affix various components in a defined relationship or otherwise configure an item for the procedure at hand.

Surgical wrenches used for this procedure are generally standard open-end wrenches which make tightening and loosening difficult due to the requirement of large radially movements radially to position the wrench and the wrench being difficult to keep in position. Therefore, there arises a need for an easy to use device for use in surgical procedures which allows for tightening or loosening of a surgical implant quickly and effectively.

### SUMMARY OF THE INVENTION

A surgical wrench includes an elongated body having a longitudinal axis, a proximal portion and an opposing distal portion. The body includes an inner shaft and a coaxial outer shaft surrounding at least a portion of the inner shaft. A removable adaptor is operatively connected to the distal portion of the body including a ratchet mechanism configured to rotate a surgical implant. A plunger is operatively associated with a proximal end of the inner shaft configured to translate the inner shaft along the central axis to engage or disengage the surgical implant.

A spring is positioned proximate the proximal end of the elongated body operatively associated with the plunger and inner shaft. Pressing the plunger towards the distal end translates the inner shaft towards the distal end. A handle encircles at least a portion of the outer shaft configured for handling and effecting rotational movement of the wrench. Pressing the plunger advances the adapter to engage or disengage with a surgical implant and releasing the plunger allows for rotational movement of the wrench by way of the handle.

A cap is disposed at the proximal end of the elongated body. A distal portion of the cap is positioned internally of the handle and surrounds the proximal end of outer shaft and a proximal portion of the cap is positioned external of the handle and adjacent the plunger.

The ratchet mechanism of the adaptor includes a ratchet ring disposed between a housing and a cover. The ratchet ring is adapted and configured to engage and disengage with the spinal implant. The housing includes a locking pin for slideably engaging the adaptor within an engagement path of the outer shaft of the elongated body. The housing includes at least one pawl arm configured to mate with the ratchet teeth of the ratchet ring and facilitate one-directional rotational movement of the ratchet ring.

The distal portion of the outer shaft includes a crossing path parallel to the central axis and an engagement path positioned distally of the crossing path. The engagement path is perpendicular to the crossing path and is configured to slideably receive a locking pin of the adapter therein.

The surgical wrench further includes a locking collar operatively connected to the distal portion of the outer shaft configured to secure the adapter with the wrench. The locking collar includes a locking tab disposed along an interior surface. The locking tab is configured to slideably engage within the crossing path to prevent the adapter from premature release from the engagement path. A compression spring is disposed between the outer shaft and the locking collar configured to allow for retracting the locking collar to expose the distal end of the outer shaft.

These and other features of the guide wire of the subject invention and the manner in which it is manufactured and employed will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 is a perspective view of an exemplary embodiment of a surgical wrench constructed in accordance with the present disclosure, showing an inner and outer shaft, and a handle;

FIG. 2 is a detailed perspective view of a distal portion of the inner and outer shafts of the wrench of FIG. 1;

FIG. 3 is a top plan view of the wrench of FIG. 1;

FIG. 4 is a cross-sectional view of the wrench of FIG. 1;

FIG. 5 is an exploded perspective view of an embodiment of a hex adaptor for use with the surgical wrench of FIG. 1, showing a ratchet mechanism;

FIG. 6 is a perspective view of the assembled hex adaptor of FIG. 5;

FIG. 7 is a perspective view of the hex adapter of FIG. 5 operatively associated with the wrench of FIG. 1;

FIG. 8 is a perspective view of an embodiment of a ratchet mechanism of a driver hex adapter for use with the surgical wrench of FIG. 1;

FIG. 9 is an exploded perspective view of an embodiment of a driver hex adapter for use with the surgical wrench of FIG. 1, showing the ratchet mechanism of FIG. 8 between a housing and a cover;

FIG. 10 is a perspective view the ratchet wrench of FIG. 1, showing a locking collar on the distal portion thereof;

FIG. 11 is a perspective view of the locking collar, showing a locking tab;

FIG. 12 is a side view of the distal end of the wrench with the locking collar in a retracted position, showing engagement of the hex adapter with the wrench of FIG. 1; and

FIG. 13 is a side view of the distal end of the wrench with the locking collar in a locked position, showing the hex adapter secured with the wrench of FIG. 1.

### DETAILED DESCRIPTION OF THE PREFERRED EMBODIMENTS

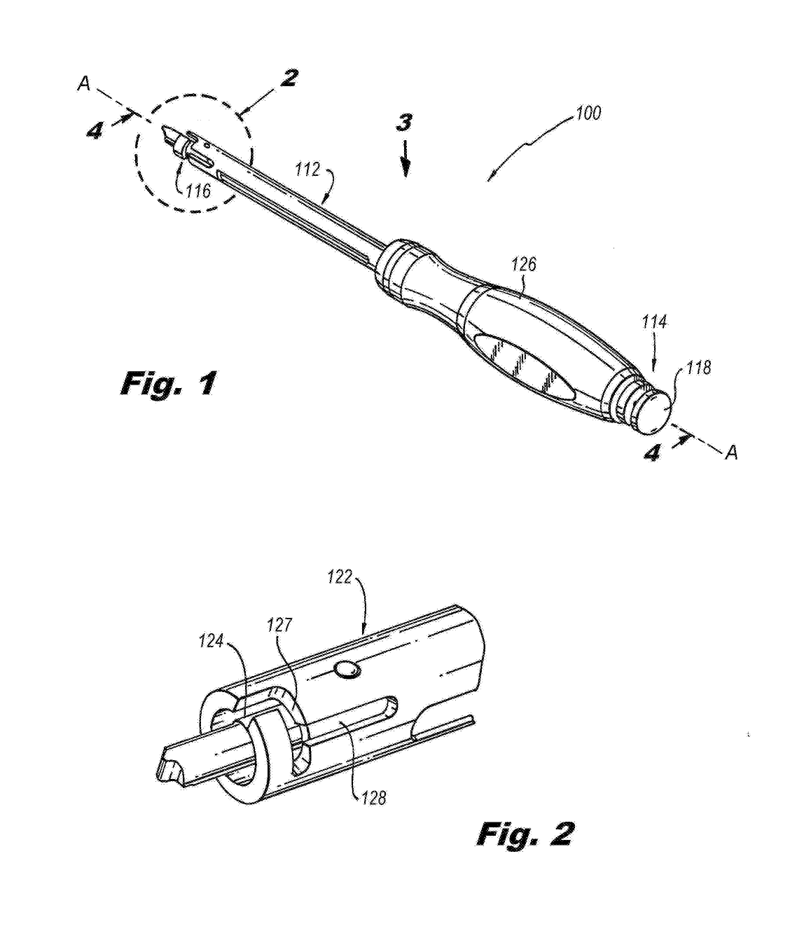

Referring now to the drawings wherein like reference numerals identify similar structural features of the claimed invention, there is illustrated in FIG. 1 a surgical wrench in accordance with a preferred embodiment of the subject invention and designated generally by reference numeral 100.

The surgical wrench 100 allows a surgeon an easier method of tightening and loosening surgical devices, for example, a hex nut of a spinal implant. An example of a spinal implant is shown and described in commonly assigned U.S. Pat. No. 9,314,276 to Hess et al., U.S. Pat. Pub. No. 2014/0194930 to Hess et al., and U.S. patent application Ser. No. 15/159,189 to Rogers et al., the disclosures of which are incorporated herein by reference in their entirety.

With reference to FIG. 1, a surgical wrench 110 is shown having an elongated body 112 with a longitudinal axis A. The body 112 includes a proximal portion 114 and opposing distal portion 116. The body 112 further includes an inner shaft 124 and an outer shaft 122 (shown best in FIG. 4) surrounding at least a portion of the inner shaft 124. The inner shaft 124 is configured to translate along the central axis A to aid in attachment with the surgical implant. A handle 126 is disposed near the proximal end 114 of the body 112 encircling at least a portion of the outer shaft 122 and is configured to effect rotational movement of the wrench 100.

As seen in FIGS. 1-4, the handle 126 is ergonomically designed to allow the surgeon control, flexibility and comfort. The diameter of the handle 126 varies from a distal end to the proximal end to allow for easily gripping the wrench. Internally, the handle 126 varies from being adjacent the outer shaft 122 to being a distance away from the outer shaft 122 with a hollow space therebetween, as best seen in FIG. 4.

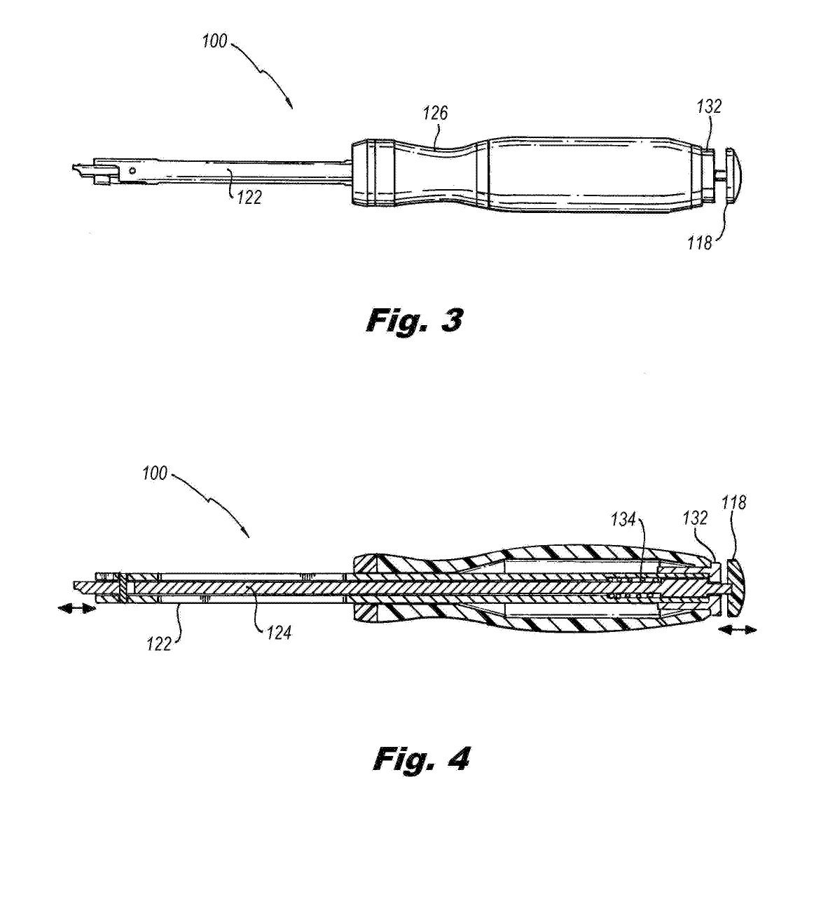

A plunger 118 is positioned at the proximal end 114 of the body 112 and is operatively associated with the inner shaft 124. A spring 134 is positioned between the inner 134 and outer 122 shaft at the proximal end 114 (shown in FIG. 3). The plunger 118 cooperates with the spring 134 to translate the inner shaft 124 along the central axis. More specifically, as the plunger 118 is pushed towards the distal end 116 the inner shaft is moved towards the distal end 116. Conversely, as the plunger 118 is released the inner shaft 124 is retracted towards the proximal end 114. The movement of the inner shaft allows the wrench to engage with the spinal implant as is described in further detail below. The spring 134 is biased in an uncompressed position such that release of the plunger 118 brings the inner shaft 124 to a neutral position.

A cap 132 is disposed at the proximal end 114 between the handle 126 and the plunger 118. A distal portion of the cap 132 rests within the hollow space of the handle 126 as shown in FIG. 4) and a proximal portion of the cap 132 is exposed adjacent the handle 126 and plunger 118. The distal portion of the cap 132 surrounds a proximal portion of the outer shaft 122. In some embodiments, the cap 132 may be threaded onto outer shaft 122 whereas in other embodiments, the cap 132 may be snap fit around the outer shaft 124.

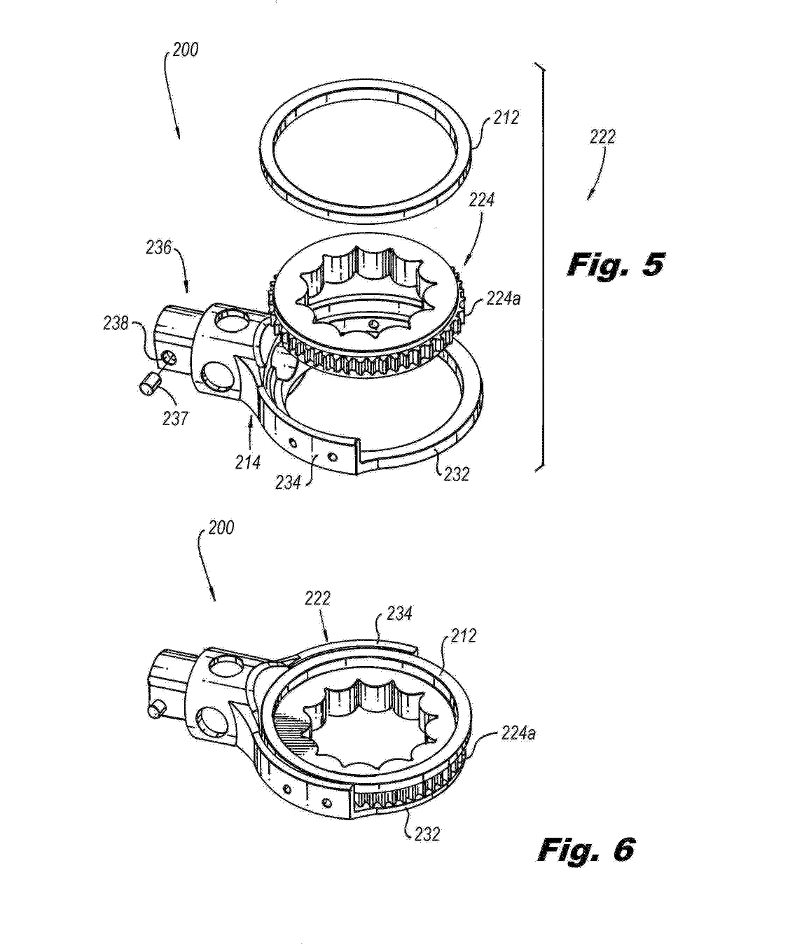

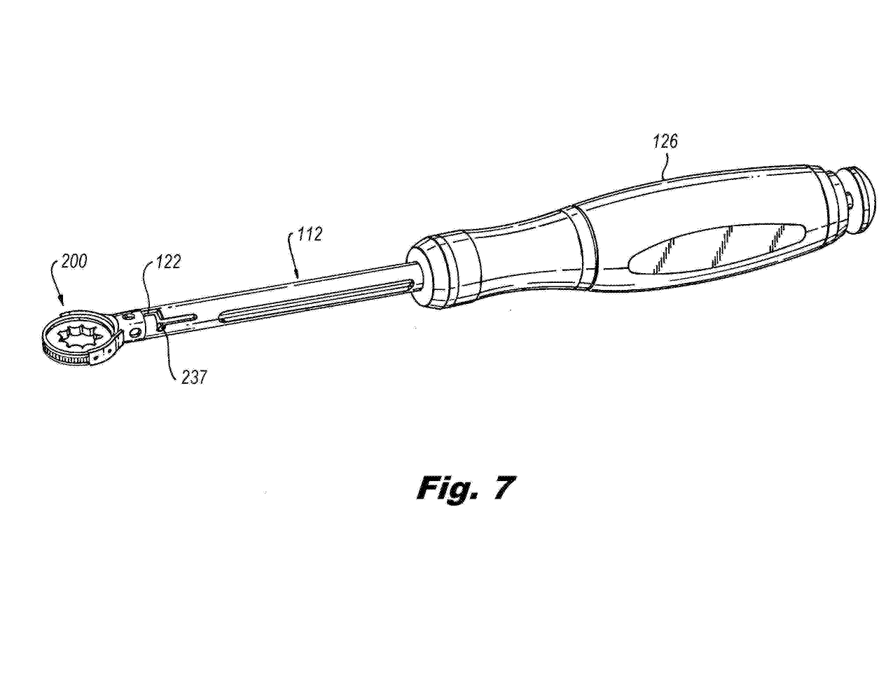

With reference to FIG. 2 a detailed view of the distal portion of the inner and outer shafts 124, 122 is shown. The wrench 100 uses modular adaptors, for example hex adapters 200 and implant drivers 300, in various sizes, connected to the body 112 to tighten or loosen a surgical implant. The outer shaft 124 includes an engagement path 127 for receiving a locking pin 237 (shown in FIGS. 5 and 7) of the hex adapter 200 therein. The locking pin 237 is slid into the engagement path 127 and the wrench 100 is rotated to secure the locking pin 237 with the wrench 100.

One embodiment of a hex adapter 200 is shown in FIGS. 5 and 6. The adapter 200 includes a ratchet mechanism 222 to rotate the surgical implant. The ratchet mechanism 222 includes a ratchet ring 224 having a plurality of ratchet teeth 224a disposed between a housing 214 and a cover 212. The ratchet mechanism 222 allows a surgeon to tighten or loosen the hex nut on an implant through small incisions and operates as a ratchet wrench but in a manual fashion. The hex adapter 200 shown can vary in size from 8 mm to 16 mm depending on the hex nut and/or surgical implant. However, those skilled in the art will readily appreciate that additional variations in sizes can be included without departing from the scope of the invention

The housing 214 includes a ring 232 and a post 236 extending outwardly from the ring 232. The post 236 includes a hole 238 for receiving the locking pin 237 therein. As shown in FIG. 5, the locking pin 237 can slideably engage with the hole 238. As best seen in FIG. 7, the locking pin 237 extends from the post 237 to mate with the engagement path 127 of the outer shaft 124 of the body 112. At least one pawl arm 234 is extends from the post 236 partially around the ring 232 of the housing 214. The pawl arm 234 allows for one-directional rotational movement of the ratchet ring 272.

The wrench 100 operates by pressing the plunger 118 thereby translating the inner shaft 124 to engage the teeth 224a with the surgical implant which provides the ratchet style operation. When a user is not pressing the plunger 118, the inner shaft 124 is returned to a neutral position and the handle 126 is rotatable relative to the surgical implant in order to grab the implant again and repeat the steps to continue tightening or loosening. There is no actual ratchet sound during use since the ratchet teeth 224a are either fully engaged or fully disengaged.

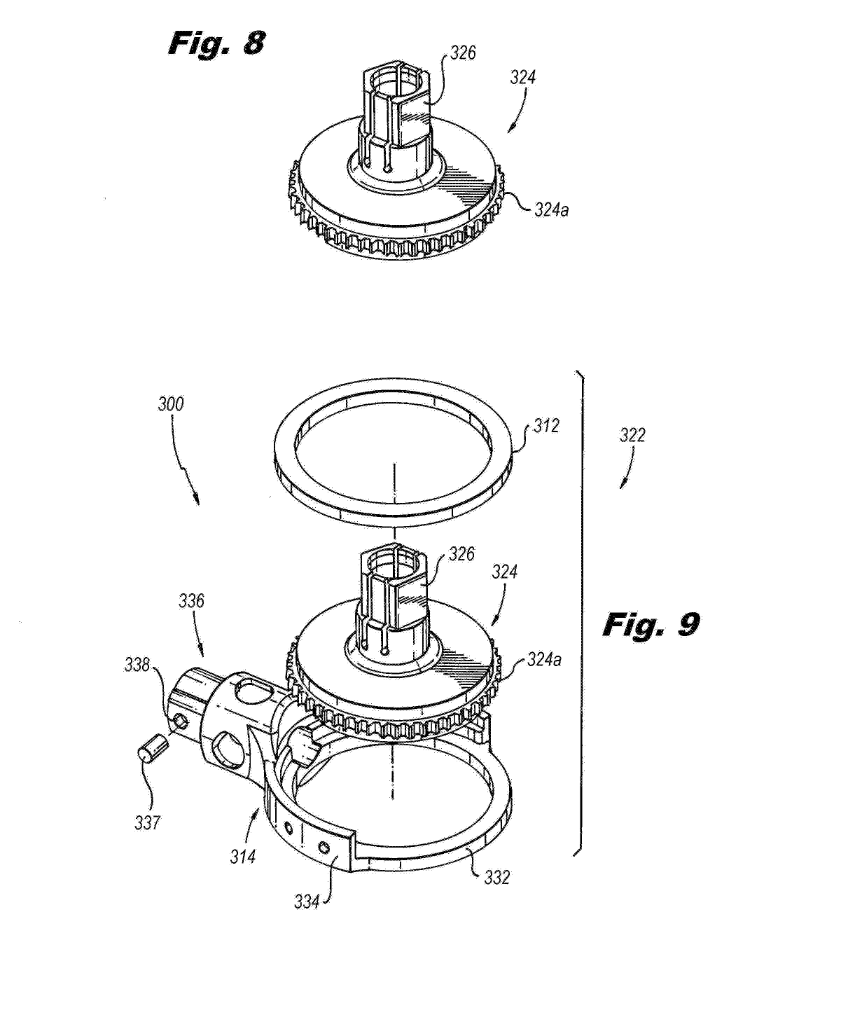

With reference to FIGS. 8 and 9 a driver hex adapter is shown for use with the surgical wrench. The driver adapter 300 is similar to adapter 200 and includes a ratchet mechanism 322. The ratchet mechanism includes a ratchet wheel 324 with a driver 326 and ratchet teeth 324a. The ratchet wheel 324 is positioned between a cover 312 and a housing 314.

Similar to housing 214, housing 314 includes a ring 332 and outwardly extending post 336. The post includes a hole 338 for accepting a locking pin 337 therein. Locking pin 337 is configured to mate with wrench 100 through engagement path 127. At least one pawl arm 334 extends from post 336 partially around ring 332 and operates in the same manner as pawl arm 234.

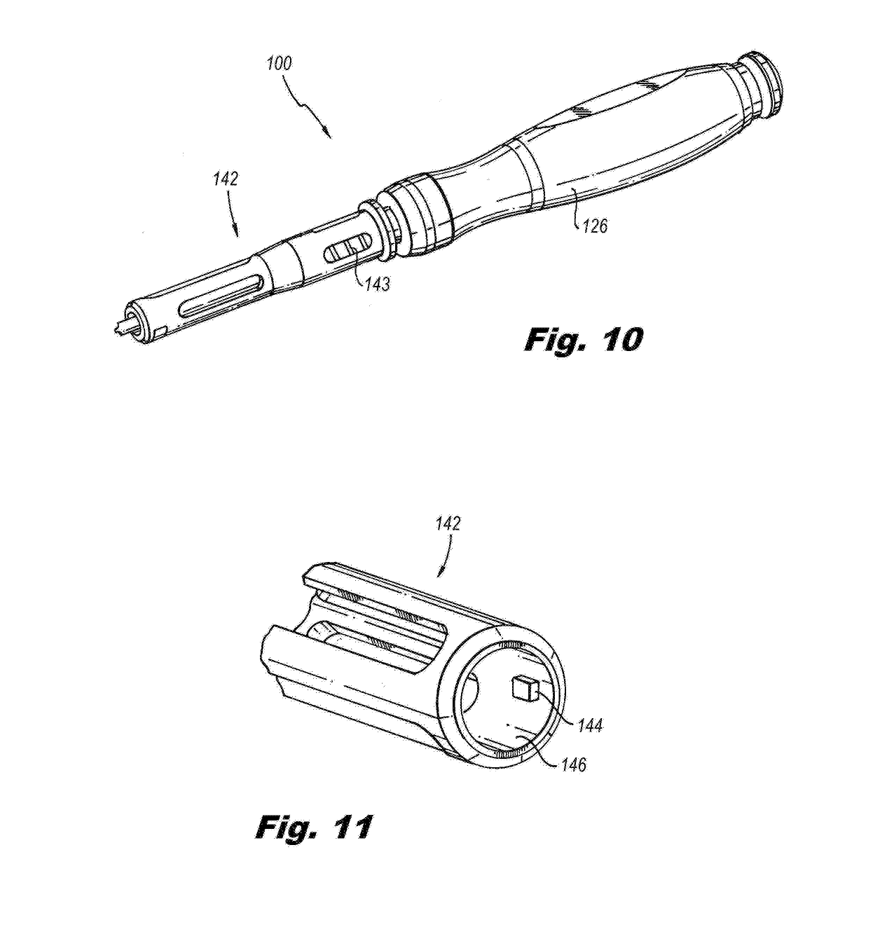

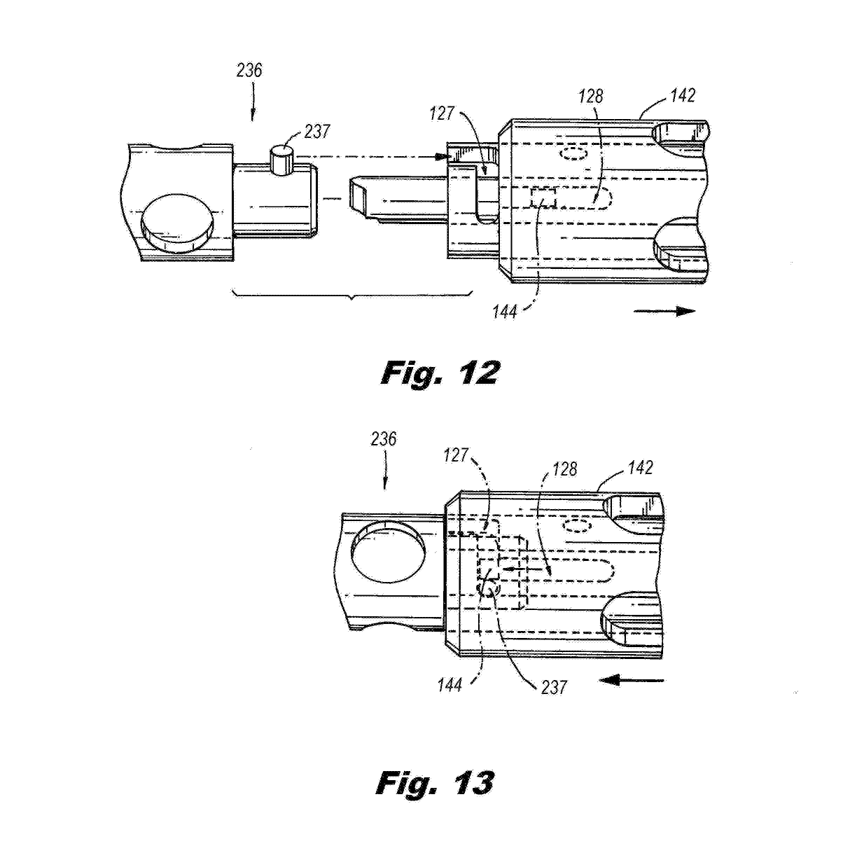

Referring now to FIG. 10, a locking collar 142 is shown over the outer shaft 122 of the wrench 100. The locking collar 142 maintains the modular adapters to the wrench 100 when the wrench 100 is in use. The locking collar 142 is a compact mechanism that fits with the profile of the wrench 100 with an easy to use assembly. The collar 142 slides over the outer shaft 122 and is operatively connected to a compressing spring 143 positioned between the outer shaft 122 and collar 142. To assemble or disassemble an adapter, e.g. 200 and 300, the collar 142 is retracted exposing the distal portion 116 of the inner and outer shafts 124, 122. The locking pin, e.g, 237 and 337, is inserted or removed from the engagement path, as described above (best seen in FIG. 12).

The collar 142 includes a locking tab 144 on an interior surface 146. The tab 144 is configured to slideably engage a crossing path 128 of the outer shaft. More specifically, as best seen in FIGS. 2 and 12 the distal portion 116 of the outer shaft 124 includes a crossing path 128 parallel to the central axis. The engagement path 127 is positioned distally of the crossing path 128 and is perpendicular thereto.

As shown in FIG. 12, once the locking pin 237 of adapter 200 is secured within the engagement path 127, the collar 142 is released and returned to its neutral position. In doing so, the locking tab 144 slides distally within the crossing path 128 and rests at an intersection of the engagement path 127 and crossing path 128. In this manner, the tab 144 secures the pin 237 in position thereby securing the adapter 200 to the wrench. The locking collar 142 is robust enough to not release the adapter 200 and more specifically, the pin 237, prematurely during manipulation and use of the wrench 100.

While the subject invention has been shown and described with reference to preferred embodiments, those skilled in the art will readily appreciate that various changes and/or modifications may be made thereto without departing from the spirit and scope of the subject invention as defined by the appended claims.

What is claimed is:

1. A surgical wrench, comprising:
an elongated body having a longitudinal axis, a proximal portion and an opposing distal portion, the body including an inner shaft and a coaxial outer shaft surrounding at least a portion of the inner shaft;
a removable adaptor operatively connected to the distal portion of the body including a ratchet mechanism configured to rotate a surgical implant; and
a plunger operatively associated with a proximal end of the inner shaft configured to translate the inner shaft along the central axis to engage or disengage the surgical implant.

2. The surgical wrench of claim 1, further comprising a spring positioned proximate the proximal end of the elongated body operatively associated with the plunger and inner shaft wherein pressing the plunger towards the distal end translates the inner shaft towards the distal end.

3. The surgical wrench of claim 2, further comprising a handle encircling at least a portion of the outer shaft configured for handling and effecting rotational movement of the wrench.

4. The surgical wrench of claim 3, wherein pressing the plunger advances the adapter to engage or disengage with a surgical implant and releasing the plunger allows for rotational movement of the wrench by way of the handle.

5. The surgical wrench of claim 3, further comprising a cap disposed at the proximal end of the elongated body, wherein a distal portion of the cap is positioned internally of the handle and surrounds the proximal end of outer shaft and a proximal portion of the cap is positioned external of the handle and adjacent the plunger.

6. The surgical wrench of claim 1, wherein the ratchet mechanism of the adaptor includes a ratchet ring disposed between a housing and a cover, the ratchet ring adapted and configured to engage and disengage with the spinal implant.

7. The surgical wrench of claim 6, wherein the housing includes a locking pin for slideably engaging the adaptor within an engagement path of the outer shaft of the elongated body.

8. The surgical wrench of claim 6, wherein the housing includes at least one pawl arm configured to mate with the ratchet teeth of the ratchet ring and facilitate one-directional rotational movement of the ratchet ring.

9. The surgical wrench of claim 1, wherein the distal portion of the outer shaft includes a crossing path parallel to the central axis and an engagement path positioned distally of the crossing path, the engagement path being perpendicular to the crossing path, wherein the engagement path is configured to slideably receive a locking pin of the adapter therein.

10. The surgical wrench of claim 9, further comprising a locking collar operatively connected to the distal portion of the outer shaft configured to secure the adapter with the wrench.

11. The surgical wrench of claim 10, wherein the locking collar includes a locking tab disposed along an interior surface configured to slideably engage within the crossing path, wherein the tab is configured to prevent the adapter from premature release from the engagement path.

12. The surgical wrench of claim 11, further comprising a compression spring disposed between the outer shaft and the locking collar configured to allow for retracting the locking collar to expose the distal end of the outer shaft.

13. A surgical wrench, comprising:
an elongated body having a central axis, a proximal portion and an opposed distal portion, the elongated body including an inner shaft and an outer shaft surrounding at least a portion of the inner shaft;
a removable adapter positioned at a distal end of the inner shaft including a ratchet ring disposed within a housing configured to engage and disengage with a spinal implant;
a locking collar operatively connected to the outer shaft configured to secure the adapter within the wrench; and
a push plunger operatively associated with a proximal end of the inner shaft configured to translate the inner shaft along the central axis, wherein pressing the plunger advances the adaptor to engage or disengage with a surgical implant and releasing the plunger allows for rotational movement of the wrench.

14. The surgical wrench of claim 13, further comprising a spring positioned approximate the proximal end of the elongated body operatively associated with the plunger and inner shaft wherein pressing the plunger towards the distal end compresses the spring to translate the inner shaft towards the distal end.

15. The surgical wrench of claim 13, further comprising a handle encircling the outer shaft configured for handling and rotational movement of the wrench.

16. The surgical wrench of claim 13, wherein the housing includes at least one pawl arm configured to mate with ratchet teeth of the ratchet ring and facilitate one-directional rotational movement of the ratchet ring.

17. The surgical wrench of claim 13, wherein the distal portion of the outer shaft includes a longitudinal crossing path and an engagement path positioned distally of the crossing path, the engagement path being perpendicular to the crossing path, wherein the engagement path is configured to slideably receive a locking pin of the adapter therein.

18. The surgical wrench of claim 17, wherein the locking collar includes a locking tab disposed along an interior surface configured to slideably engage within the crossing path, wherein the locking tab is configured to prevent the adapter from exiting the engagement path.

19. A manual wrench for tightening and loosening a surgical implant, comprising:
an elongated body having a central axis, a proximal portion and an opposed distal portion, the elongated body including an inner shaft and an outer shaft surrounding at least a portion of the inner shaft;
a removable adapter positioned at a distal end of the inner shaft configured to rotate a spinal implant; and
a push plunger operatively associated with a proximal end of the inner shaft configured to translate the inner shaft along the central axis, wherein pressing the plunger advances the adaptor to engage or disengage a the surgical implant and releasing the plunger allows for rotational movement of the wrench.

20. The manual wrench of claim 19, wherein the adapter includes a ratchet mechanism including a ratchet ring disposed between a housing and a cover, the ratchet ring adapted and configured to rotate the surgical implant.
